(19) United States
(12) Patent Application Publication
(10) Pub. No.: US 2005/0084488 A1
(43) Pub. Date: Apr. 21, 2005

(54) Asparagine deaminase catalytic antibodies

(76) Inventors: Michael G. Mulkerrin, Hillsborough, CA (US); Ruoheng Zhang, Fremont, CA (US); Lorin Roskos, Pleasonton, CA (US); Peter Blumbergs, Royal Oak, MI (US); Dumitru Lonescu, Ann Arbor, MI (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/821,626

(22) Filed: Apr. 9, 2004

Related U.S. Application Data
(60) Provisional application No. 60/462,550, filed on Apr. 10, 2003.

(51) Int. Cl. A61K 39/395; C12N 9/00; C07F 9/28

(52) U.S. Cl. 424/130.1; 435/188.5; 530/387.1; 562/14

(57) ABSTRACT

Transition state analogs are described which may be used to elicit antibodies that catalyze the conversion of asparagine to aspartic acid. Synthetic schemes are disclosed for making the transition state analogs which can than be attached to a carrier molecule to form an immunoconjugate. Immunoconjugates can be administered to an animal for the purpose of raising antibodies. Antibodies can in turn be used in pharmaceutical compositions which can be given to patients as part of a method of treating various conditions, particularly cancer.

### RELATED APPLICATIONS

This application claims priority to, and hereby incorporates the entire disclosure of, co-pending U.S. provisional application No. 60/462,550, filed Apr. 10, 2003, and entitled “ASPARAGINE DEAMINASE CATALYTIC ANTIBODIES.”

### BACKGROUND OF THE INVENTION

1. Field of the Invention

The present invention is generally directed to the field of catalytic antibodies, and particularly directed to antibodies for catalyzing the conversion of asparagine to aspartic acid, transition state analogs for making the antibodies, and methods of using the antibodies. The antibodies can be administered to a patient and can be useful for treating diseases such as acute lymphoblastic leukemia, chronic lymphocytic leukemia, Hodgkin's disease, Non-Hodgkin's disease and multiple myeloma.

2. Description of the Related Art

One current treatment for hematopoietic cancers such as acute lymphoblastic leukemia is the administration to patients of the enzyme asparagine deaminase (AD). AD catalyses the conversion of asparagine to aspartic acid. This conversion lowers the circulating concentration of asparagine in the body, rendering tumor cells susceptible to apoptosis, and hence assists in the treatment of the disease.

As the source of AD is non-human (typically obtained from E. coli) it has significant instances of adverse immune reactions in patients. The titre of AD in a patient must be carefully monitored because of this side effect as well as others such as coagulation disorders, azotemia, liver function abnormalities, and the like.

As such, there exits a need of therapeutic agents that catalyze the conversion of asparagine to aspartic acid in a patient, especially so in one suffering cancer, that has a more favorable side effect profile. Furthermore, it is desirable that such a therapeutic agent be able to be manufactured in a relatively easy and reproducible way. The instant invention provides for these needs and has other advantages as well.

### SUMMARY OF THE INVENTION

One aspect of the invention is a transition state analog having the following structure: 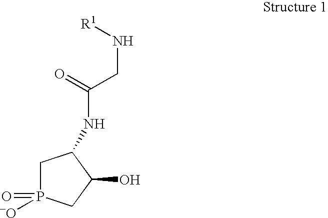
wherein R₁ is a hydrogen atom, an amino protecting group or an immunoconjugate carrier molecule. This aspect of the invention can also include salts and esters thereof.

Another aspect of the invention is a monoclonal catalytic antibody specific for a transition state analog of Structure 1 that catalyzes the deamidation of asparagine to aspartic acid.

Another aspect of the invention is a pharmaceutical composition containing a pharmaceutically acceptable amount of the monoclonal catalytic antibody above and one or more pharmaceutically acceptable carriers therefor.

Another aspect of the invention is a method of treating cancer which includes administering to a patient in need of such treatment a pharmaceutically effective amount of the above pharmaceutical composition.

Another aspect of the invention is a method of catalyzing the deamidation of asparagine to aspartic acid including contacting the asparagine-containing material with one of the above monoclonal catalytic antibodies.

Another aspect of the invention is a method of expressing the above monoclonal catalytic antibodies that includes immunizing a mouse with a compound of Structure 1, forming a hybridoma from the spleen of the mouse so immunized, and isolating the antibody expressed from the hybridoma.

Another aspect of the invention is a transition state analog having the following structure: 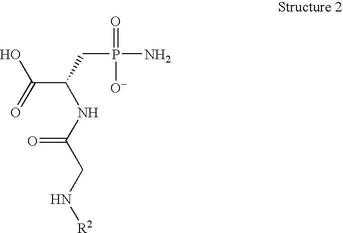
wherein R2 is a hydrogen atom, an amino protecting group or an immunoconjugate carrier molecule. This aspect of the invention can also include salts and esters thereof.

Another aspect of the invention is a monoclonal catalytic antibody specific for a transition state analog of Structure 2 that catalyzes the deamidation of asparagine to aspartic acid.

Another aspect of the invention is a pharmaceutical composition containing a pharmaceutically acceptable amount of the monoclonal catalytic antibody above and one or more pharmaceutically acceptable carriers therefor.

Another aspect of the invention is a method of treating cancer which includes administering to a patient in need of such treatment a pharmaceutically effective amount of the above pharmaceutical composition.

Another aspect of the invention is a method of catalyzing the deamidation of asparagine to aspartic acid including contacting the asparagine-containing material with one of the above monoclonal catalytic antibodies.

Another aspect of the invention is a method of expressing the above monoclonal catalytic antibodies that includes immunizing a mouse with a compound of Structure 2, forming a hybridoma from the spleen of the mouse so immunized, and isolating the antibody expressed from the hybridoma.

### DETAILED DESCRIPTION

Some embodiments of the invention include a transition state analog having the following structure: 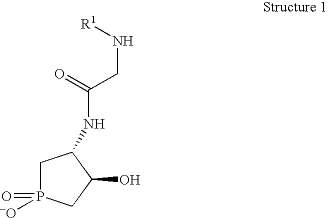
wherein R1 is a hydrogen atom, an amino protecting group or an immunoconjugate carrier molecule. Some embodiments of the invention also includes salts and esters thereof. By way of example, R1 can be a hydrogen atom, an immunoconjugate molecule, or a KLH immunoconjugate molecule.

Some embodiments of the invention include a monoclonal catalytic antibody specific for a transition state analog of Structure 1 that catalyzes the deamidation of asparagine to aspartic acid. In some embodiments, the antibody is a murine one, a human one, a human one of the IgG class, or a human one of the IgG₂ class.

Some embodiments of the invention include a pharmaceutical composition containing a pharmaceutically acceptable amount of the monoclonal catalytic antibody above and one or more pharmaceutically acceptable carriers therefor. In some embodiments, the monoclonal catalytic antibody is a murine one, a human one, a human one of the IgG class, or a human one of the IgG₂ class.

Some embodiments of the invention include a method of treating cancer which include administering to a patient in need of such treatment a pharmaceutically effective amount of the above pharmaceutical composition. In some embodiments of this invention, the monoclonal catalytic antibody used in the pharmaceutical composition is a murine one, a human one, a human one of the IgG class, or a human one of the IgG₂ class. In some embodiments the cancer to be treated is a hematopoietic one. In some embodiments, the cancer to be treated is acute lymphoblastic leukemia, chronic lymphocytic leukemia, Hodgkin's disease, Non-Hodgkin's disease, or multiple myeloma.

Some embodiments of the invention include a method of catalyzing the deamidation of asparagine to aspartic acid which include contacting the asparagine-containing material with one of the above monoclonal catalytic antibodies.

Some embodiment of the invention include a method of expressing the above monoclonal catalytic antibodies that includes immunizing a mouse with a compound of Structure 1, forming a hybridoma from the spleen of the mouse so immunized, and isolating the antibody expressed from the hybridoma.

Some embodiments of the invention include a transition state analog having the following structure: 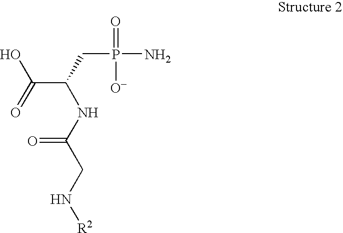
wherein R₂ is a hydrogen atom, an amino protecting group or a carrier molecule. Some embodiments can also include salts and esters thereof. By way of example, R₁ can be a hydrogen atom, an immunoconjugate molecule, or a KLH immunoconjugate molecule.

Some embodiments of the invention include a monoclonal catalytic antibody specific for a transition state analog of Structure 2 that catalyzes the deamidation of asparagine to aspartic acid. In some embodiments, the antibody is a murine one, a human one, a human one of the IgG class, or a human one of the IgG₂ class.

Some embodiments of the invention include a pharmaceutical composition containing a pharmaceutically acceptable amount of the monoclonal catalytic antibody above and one or more pharmaceutically acceptable carriers therefor. In some embodiments, the antibody is a murine one, a human one, a human one of the IgG class, or a human one of the IgG₂ class.

Some embodiments of the invention include a method of treating cancer which includes administering to a patient in need of such treatment a pharmaceutically effective amount of the above pharmaceutical composition In some embodiments, the antibody in the pharmaceutical formulation used for the method is a murine one, a human one, a human one of the IgG class, or a human one of the IgG₂ class. In some embodiments, the cancer to be treated is a hematopoietic one. In some embodiments, the cancer to be treated is acute lymphoblastic leukemia, chronic lymphocytic leukemia, Hodgkin's disease, Non-Hodgkin's disease or multiple myeloma.

Some embodiments of the invention include a method of catalyzing the deamidation of asparagine to aspartic acid comprising contacting the asparagine-containing material with one of the above monoclonal catalytic antibodies.

Some embodiments of the invention include a method of expressing the above monoclonal catalytic antibodies that includes immunizing a mouse with a compound of Structure 2, forming a hybridoma from the spleen of the mouse so immunized, and isolating the antibody expressed from the hybridoma.

As used herein, the terms “antibody” and “antibodies” encompass both whole antibodies as well as antibody fragments, such as single chain Fv.

The term “transition state analog” refers to a moiety that is capable of inducing the formation of antibodies, particularly when bound to an antigenic protein or other large carrier molecule, other references may refer to a transition state analog as a “hapten.”

The term “monoclonal catalytic antibody” refers to an antibody that catalyzes a reaction diagramed in the following Scheme 1

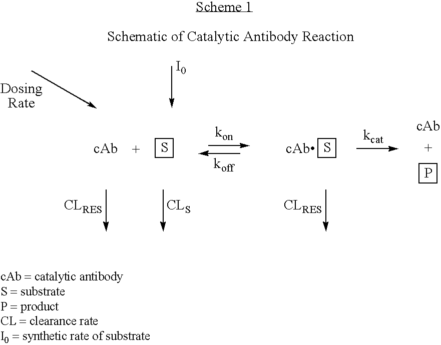

In the above Scheme 1, “cAB” are the monoclonal catalytic antibodies of the instant invention, “S” is asparagine, “P” is aspartic acid and “cAB.S” is the catalytic antibody-substrate complex. Thus, some embodiments of the present invention include monoclonal catalytic antibodies that have been raised against the transition state analog of the reaction that converts asparagine to aspartic acid. Examples of these transition state analogs are Structures 1 and 2 above.

The monoclonal catalytic antibodies of this invention will preferably have a longer half life than the asparagine deaminase enzyme currently used in cancer therapy. In some embodiments of the this invention, the affinity of the antibody for asparaginase is between about 10 and about 100 μM and a k_{cat} of between about 30 to about 200, and also about 100 to about 200/day.

The term “immunogenic conjugate” refers to a complex wherein a transition state analog is coupled with a carrier molecule such as a protein. Preferably, an immunogenic conjugate is capable of eliciting the formation of antibodies when administered to an animal.

The term “keyhole limpet hemocyanin” (“KLH”) refers to a carrier molecule to which a transition state analog can be coupled. KLH is commercially available and its source is typically the hemolymphs of the mollusk, Megathura crenulata. Although KLH is a preferred carrier molecule for many embodiments of the present invention, references to KLH herein are intended to be illustrative and not limiting as other suitable carrier molecules can be used as alternatives.

As used herein, the term “animal” includes live animals as well as cell lines from animals which are sustained artificially. Hence, a reference to inducing an animal to produce antibodies and harvesting the antibodies includes inducing and harvesting antibodies using an in vitro cell line.

The following reaction scheme shows the deamidation of asparagine to aspartic acid by way of a cyclic intermediate:

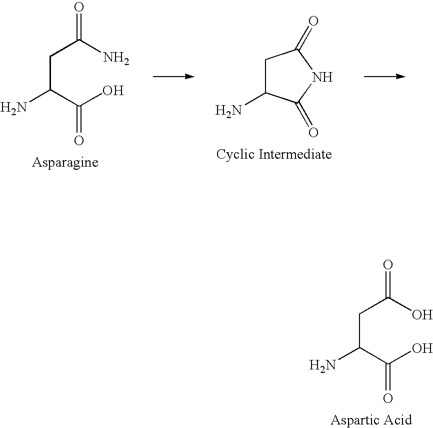

The cyclic intermediate in this reaction is generally an unstable, high-energy species. For this reason, the progression from asparagine to aspartic acid is slow. Some embodiments of the present invention provide an antibody which can form a complex with the intermediate, thereby stabilizing it, and effectively accelerate the conversion of asparagine to aspartic acid.

A transition state analog according to Structure 1 or Structure 2 can be coupled to a carrier molecule, such as KLH. For Structure 1, binding to KLH preferably occurs at the glycyl (acetylated) terminal nitrogen. For Structure 2, binding to KLH preferably occurs at the terminal primary amino group.

As noted above, an immunogen is prepared by coupling the transition state analogs of FIGS. 1 and 2 to an immunoconjugate carrier molecule. Useful carrier molecules are proteins such as keyhole limpet hemocyanin (KLH), edestin, albumins, such as bovine or human serum albumin (BSA or HSA), tetanus toxoid, and cholera toxoid, polyaminoacids, such as poly(D-lysine-D-glutamic acid), as well as red blood cells, such as sheep erythrocytes (SRBC).

The immune system of the animal to which the immunogenic conjugate has been administered can then begin producing antibodies to the immunogenic conjugate. Typically, the immune system generates many types of antibodies in such a response. Those antibodies which are specific to the transition state analog are generally the most useful and can be screened and isolated using standard techniques in the art. As antibodies can be modified in a number of ways, the term “antibody” should be construed as covering any specific binding member or substance having a binding domain with the required specificity and catalytic activity. Thus, this term covers antibody fragments, derivatives, functional equivalents and homologues of antibodies, including any polypeptide comprising an immunoglobulin binding domain, whether natural or wholly or partially synthetic. Monoclonal antibodies are preferred antibodies; accordingly, methods of preparation and isolation of such antibodies are preferred techniques. Fragments of antibodies can also be used. Examples of such antibody fragments include ScFv, Fab₂, Fv, etc.

Monoclonal antibodies to the transition-state analogs can be prepared using any technique that provides for the production of antibody molecules by continuous cell lines in culture. These include, but are not limited to, the hybridoma technique originally described by Koehler et al. (1975) Nature 256:495-497, the human B-cell hybridoma technique (Kosbor et al. (1983) Immunol Today 4:72; Cote et al. (1983) Proc Natl Acad Sci 80:2026-2030), and the EBV-hybridoma technique (Cole et al. Monoclonal Antibodies and Cancer Theraipy, Alan R. Liss Inc, New York, 77-96 (1985). In addition, techniques developed for the production of “chimeric antibodies,” the splicing of mouse antibody genes to human antibody genes to obtain a molecule with appropriate antigen specificity and biological activity can be used. (Morrison et al. (1984) Proc Natl Acad Sci 81:6851-6855; Neuberger et al. (1984) Nature 312:604-608; Takeda et al. (1985) Nature 314:452-454). Alternatively, techniques described for the production of single chain antibodies (U.S. Pat. No. 4,946,778 (Ladner et al.), hereby expressly incorporated by reference in its entirety) can be adapted to produce anti-transition-state analog single chain catalytic antibodies. Catalytic antibodies can also be produced by inducing in vivo production in the lymphocyte population or by screening recombinant immunoglobulin libraries or panels of highly specific binding reagents as disclosed in Orlandi et al. (1989) Proc Natl Acad Sci 86: 3833-3837, and Winter et al. (1991) Nature 349:293-299, both of which are hereby expressly incorporated by reference in their entireties.

In some embodiments, a KLH conjugated TSA (for example of FIG. 1 or FIG. 2) is used to immunize XenoMouse® mice, available from Abgenix (e.g., Mendez et al. Nat. Genet. February 1997;15(2):146-56 and U.S. patent application Ser. No. 08/464,584, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,859, filed Jun. 5, 1995, and Ser. No. 08/759,620, filed Dec. 3, 1996 and U.S. Pat. Nos. 5,939,598, 6,075,181, 6,114,598, 6,150,584, 6,162,963, 6,235,883 and 6,673,986, 6,682,736, 6,713,610, and Japanese Patent Nos. 3 068 180 B2, 3 068 506 B2, and 3 068 507 B2 and European Patent No., EP 0 463 151 B1, grant published June 12, 1996, International Patent Application No., WO 94/02602, published Feb. 3, 1994, International Patent Application No., WO 96/34096, published October 31, 1996, WO 98/24893, published June 11, 1998, WO 00/76310, published December 21, 2000. The disclosures of each of the above-cited patents, applications, and references are hereby incorporated by reference in their entirety). Immunization can be accomplished using standard techniques and protocols. Immune response in the animals to the TSA can be measured, for example through measuring serum titers of anti-TSA antibodies. When desired levels of immune response are achieved, mice are sacrificed and B cells recovered which can be utilized to generate hybridomas, using standard protocols and techniques, or used directly to identify B cells producing antibodies of the desired function. Further techniques for generating antibodies by the use of B cells are discussed in U.S. Pat. No. 5,627,052 (Schrader) and Babcook et al. Proc Natl Acad Sci USA. Jul. 23, 1996;93(15):7843-8, which include techniques and products which are sometimes referred to as Xenomax®. Both references are hereby incorporated by reference in their entireties.

In some embodiments, antibody producing cells (e.g., hybridomas, B cells, or recombinant lines prepared therefrom) are utilized to assay for antibodies that bind to the TSA and which further are capable of catalyzing deamidation of asparagine to aspartic acid, as described herein. Upon identifying antibodies that are capable of catalyzing deamidation of asparagine to aspartic acid, if hybridoma derived, quantities of the antibodies can be expressed or the heavy and light chain genes encoding such antibodies can be cloned from the hybridoma or B cell into expression vectors and used to prepare recombinant cell lines expressing the antibodies using common protocols and techniques.

Catalytic antibody fragments to the transition-state analogs thereof can also be generated. For example, such fragments include, but are not limited to, the F(ab′)₂ fragments that can be produced by pepsin digestion of the antibody molecule and the Fab fragments that can be generated by reducing the disulfide bridges of the F(ab′)₂ fragments. Alternatively, Fab expression libraries can be constructed to allow rapid and easy identification of monoclonal Fab fragments with the desired specificity. (Huse et al. (1989) Science 256:1275-1281), U.S. Pat. No. 5,439,812, both of which are hereby expressly incorporated by reference in their entireties.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward et al. (1989) Nature 341:544-546) which consists of a VH domain; (v) isolated CDR regions; (vi) F(ab′)2 fragments, a bivalent fragment comprising two linked Fab fragments; (vii) single chain Fv molecules (scFv), wherein a VH domain and a VL domain are linked by a peptide linker which allows the two domains to associate to form an antigen binding site (Bird et al. (1988) Science 242:423-426; Huston et al. (1988) Proc Natl Acad Sci USA 85:5879-5883); (viii) bispecific single chain Fv dimers (PCT/US92/0996S); and (ix) “diabodies,” multivalent or multispecific fragments constructed by gene fusion (WO94/13804 (Holliger et al.); Holliger et al. (1993) Current Opinion Biotechnol 4:446-449), both of which are hereby incorporated by reference in their entireties.

Diabodies are multimers of polypeptides, each polypeptide comprising a first domain comprising a binding region of an immunoglobulin light chain and a second domain comprising a binding region of an immunoglobulin heavy chain, the two domains being linked (e.g., by a peptide linker) but unable to associate with each other to form an antigen binding site: antigen binding sites are formed by the association of the first domain of one polypeptide within the multimer with the second domain of another polypeptide within the multimer (WO94/13804 (Holliger et al.)), hereby incorporated by reference in its entirety.

Where bispecific antibodies are to be used, these may be conventional bispecific antibodies, which can be manufactured in a variety of ways (Holliger et al. (1993) supra), e.g., prepared chemically or from hybrid hybridomas, or may be any of the bispecific antibody fragments mentioned above. It may be preferable to use scFv dimers or diabodies rather than whole antibodies. Diabodies and scFv can be constructed without an Fc region, using only variable domains, potentially reducing the effects of anti-idiotypic reaction. Other forms of bispecific antibodies include the single chain “Janus ins” described in Traunecker et al. (1991) Embo J 10:3655-3659, which is hereby incorporated by reference in its entirety.

Bispecific diabodies, as opposed to bispecific whole antibodies, may also be particularly useful because they can be readily constructed and expressed in E.coli. Diabodies (and many other polypeptides such as antibody fragments) of appropriate binding specificities can be readily selected using phage display (WO94/13804 (Holliger et al.) from libraries. If one arm of the diabody is to be kept constant, for instance, with a specificity directed against antigen X, then a library can be made where the other arm is varied and an antibody of appropriate specificity selected.

Antibody Structure

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site.

Thus, an intact antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann Clin. Exp. Immunol. 79: 315-321 (1990), Kostelny et al: J. Immunol. 148:1547-1553 (1992). Production of bispecific antibodies can be a relatively labor intensive process compared with production of conventional antibodies and yields and degree of purity are generally lower for bispecific antibodies. Bispecific antibodies do not exist in the form of fragments having a single binding site (e.g., Fab, Fab′, and Fv).

Human Antibodies and Humanization of Antibodies

Human antibodies avoid certain of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, it has been postulated that one can develop humanized antibodies or generate fully human antibodies through the introduction of human antibody function into a rodent so that the rodent would produce fully human antibodies.

The ability to clone and reconstruct megabase-sized human loci in YACs and to introduce them into the mouse germline provides a powerful approach to elucidating the functional components of very large or crudely mapped loci as well as generating useful models of human disease. Furthermore, the utilization of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (Mabs)—an important milestone towards fulfilling the promise of antibody therapy in human disease. Fully human antibodies are expected to minimize the immunogenic and allergic responses intrinsic to mouse or mouse-derivatized Mabs and thus to increase the efficacy and safety of the administered antibodies. The use of fully human antibodies can be expected to provide a substantial advantage in the treatment of chronic and recurring human diseases, such as inflammation, autoimmunity, and cancer, which require repeated antibody administrations.

One approach towards preparing fully human antibodies was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human Mabs with the desired specificity could be readily produced and selected.

This general strategy was demonstrated in connection with the generation of the first XenoMouse™ strains as published in 1994. See Green et al. Nature Genetics 7:13-21 (1994). The XenoMouse™ strains were engineered with yeast artificial chromosomes (YACs) containing 245 kb and 190 kb-sized germline configuration fragments of the human heavy chain locus and kappa light chain locus, respectively, which contained core variable and constant region sequences. Id. The human Ig containing YACs proved to be compatible with the mouse system for both rearrangement and expression of antibodies and were capable of substituting for the inactivated mouse Ig genes. This was demonstrated by their ability to induce B-cell development, to produce an adult-like human repertoire of fully human antibodies, and to generate antigen-specific human Mabs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was recently extended to the introduction of greater than approximately 80% of the human antibody repertoire through introduction of megabase sized, germline configuration YAC fragments of the human heavy chain loci and kappa light chain loci, respectively, to produce XenoMouse™ mice. See Mendez et al. Nature Genetics 15:146-156 (1997) and U.S. patent application Ser. No. 08/759,620, filed Dec. 3, 1996, the disclosures of which are hereby incorporated by reference.

Such approach is further discussed and delineated in U.S. patent application Ser. No. 08/464,584, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,859, filed Jun. 5, 1995, and Ser. No. 08/759,620, filed Dec. 3, 1996 and U.S. Pat. Nos. 5,939,598, 6,075,181, 6,114,598, 6,150,584, 6,162,963, 6,235,883 and 6,673,986, 6,682,736, 6,713,610, and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998). See also European Patent No., EP 0 463 151 B1, grant published June 12, 1996, International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000. The disclosures of each of the above-cited patents, applications, and references are hereby incorporated by reference in their entirety.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more V_{H} genes, one or more DH genes, one or more J_{H} genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. No. 5,591,669 and 6,023,010 to Krimpenfort and Berns, U.S. Pat. Nos. 5,612,205, 5,721,367, and 5,789,215 to Berns et al., and U.S. Pat. No. 5,643,763 to Choi and Dunn, and GenPharm International, U.S. Pat. Nos. 5,545,806, 5,569,825, 5,625,126, 5,661,016, 5,770,429, 5,789,650, 5,814,318, 6,300,129, the disclosures of which are hereby incorporated by reference. See also European Patent No. 0 546 073 B1, International Patent Application Nos. WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), Fishwild et al., (1996), the disclosures of which are hereby incorporated by reference in their entirety.

The inventors of Surani et al., cited above and assigned to the Medical Research Counsel (the “MRC”), produced a transgenic mouse possessing an Ig locus through use of the minilocus approach. The inventors on the GenPharm International work cited above, Lonberg and Kay, proposed inactivation of the endogenous mouse Ig locus coupled with substantial duplication of the Surani et al. work.

An advantage of the minilocus approach is the rapidity with which constructs including portions of the Ig locus can be generated and introduced into animals. Commensurately, however, a significant disadvantage of the minilocus approach is that, in theory, insufficient diversity is introduced through the inclusion of small numbers of V, D, and J genes. Indeed, the published work appears to support this concern. B-cell development and antibody production of animals produced through use of the minilocus approach appear stunted. Therefore, research surrounding the present invention has consistently been directed towards the introduction of large portions of the Ig locus in order to achieve greater diversity and in an effort to reconstitute the immune repertoire of the animals.

Kirin has also demonstrated the generation of human antibodies from mice in which, through microcell fusion, large pieces of chromosomes, or entire chromosomes, have been introduced. See European Patent Application Nos. 773 288 and 843 961, the disclosures of which are hereby incorporated by reference.

Human anti-mouse antibody (HAMA) responses have led the industry to prepare chimeric or otherwise humanized antibodies. While chimeric antibodies have a human constant region and a murine variable region, it is expected that certain human anti-chimeric antibody (HACA) responses will be observed, particularly in chronic or multi-dose utilizations of the antibody. Thus, fully human antibodies against the instant transition state analogs that vitiate concerns and/or effects of HAMA or HACA response, as well as possess other advantages as discussed above, can be made by the procedures discussed above.

Humanization and Display Technologies

As was discussed above in connection with human antibody generation, there are advantages to producing antibodies with reduced immunogenicity. To a degree, this can be accomplished in connection with techniques of humanization and display techniques using appropriate libraries. It will be appreciated that murine antibodies or antibodies from other species can be humanized or primatized using techniques well known in the art. See e.g., Winter and Harris Immunol Today 14:43-46 (1993) and Wright et al. Crit, Reviews in Immunol. 12125-168 (1992). The antibody of interest may be engineered by recombinant DNA techniques to substitute the CH1, CH2, CH3, hinge domains, and/or the framework domain with the corresponding human sequence (see WO 92/02190 and U.S. Pat. Nos. 5,530,101, 5,585,089, 5,693,761, 5,693,792, 5,714,350, and 5,777,085). Also, the use of Ig cDNA for construction of chimeric immunoglobulin genes is known in the art (Liu et al. P.N.A.S. 84:3439 (1987) and J.Immunol.139:3521 (1987)). MRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions genes may be found in Kabat et al. (1991) Sequences of Proteins of Immunological Interest, N.I.H. publication no. 91-3242. Human C region genes are readily available from known clones. The choice of isotype will be guided by the desired effector functions, such as complement fixation, or activity in antibody-dependent cellular cytotoxicity. Preferred isotypes are IgG1, IgG3 and IgG4. Either of the human light chain constant regions, kappa or lambda, may be used. The chimeric, humanized antibody is then expressed by conventional methods.

Antibody fragments, such as Fv, F(ab′).sub.2 and Fab may be prepared by cleavage of the intact protein, e.g. by protease or chemical cleavage. Alternatively, a truncated gene is designed. For example, a chimeric gene encoding a portion of the F(ab′)₂ fragment would include DNA sequences encoding the CH1 domain and hinge region of the H chain, followed by a translational stop codon to yield the truncated molecule.

Consensus sequences of H and L J regions may be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

Expression vectors include plasmids, retroviruses, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody may be joined to any strong promoter, including retroviral LTRs, e.g. SV-40 early promoter, (Okayama et al. Mol. Cell. Bio. 3:280 (1983)), Rous sarcoma virus LTR (Gorman et al. P.N.A.S. 79:6777 (1982)), and moloney murine leukemia virus LTR (Grosschedl et al. Cell 41:885 (1985)). Also, as will be appreciated, native Ig promoters and the like may be used.

Further, human antibodies or antibodies from other species can be generated through display-type technologies, including, without limitation, phage display, retroviral display, ribosomal display, and other techniques, using techniques well known in the art and the resulting molecules can be subjected to additional maturation, such as affinity maturation, as such techniques are well known in the art. Wright and Harris, supra., Hanes and Plucthau Proc Natl Acad Sci USA 94:4937-4942 (1997) (ribosomal display), Parmley and Smith Gene 73:305-318 (1988) (phage display), Scott TIBS 17:241-245 (1992), Cwirla et al. Proc Natl Acad Sci USA 87:6378-6382 (1990), Russel et al. Nucl. Acids Research 21:1081-1085 (1993), Hoganboom et al. Immunol. Reviews 130:43-68 (1992), Chiswell and McCafferty TIBTECH 10:80-84 (1992), and U.S. Pat. No. 5,733,743. If display technologies are utilized to produce antibodies that are not human, such antibodies can be humanized as described above.

Using these techniques, antibodies can be generated to instant transition state analogs, which can thereafter be screened as described above for the activities described above as well as below.

A further means for assaying the activity of the monoclonal catalytic antibodies is to use assays currently used to assay for the activity of L-Asparaginase activity. These assays are known in the art. (See, for example, P. Ylikangas et al., Analytical Biochemistry, 280, 42-45 (2000), herein incorporated by reference.) In this method, the antibody would be incubated with L-aspartic acid (7-amido-4-methylcoumarin) and the release of 7-amino-4-methylcoumarin is measured fluorometrically. Other methods of analysis can be used, such as using L-asparaginase (LAS) sensitive human leukemia and lymphoma cell lines, determining potency of the monoclonal catalytic antibodies using colony forming assays or apoptosis assays. In vivo assays include the use of human leukemia SCID mouse xenograft models. The antibodies of the instant invention are tested in these SCID models in monotherapy or in combined therapy with prednisone, vincristine (for ALL) and cytarabine (ara-C, for myeloblastic leukemia.)

A further assessment of the catalytic activity of the instant antibodies will be the analysis of the traditional Michaelis-Menton kinetics of the antibody. HPLC techniques can be used to quantitate the amounts of the asparagine and aspartic acid in the catalytic conversion reaction. Using the velocity of the reaction at a number of initial substrate concentrations, the Km and the Vmax of the antibody can be determined.

The population of cell lines secreting a population of monoclonal antibodies is then screened to identify those cell lines which are producing antibodies that exhibit catalytic activity in the deamidation of the asparaginyl-glycyl dipeptide. This screening may be done directly for individual antibodies as described above or may be accomplished in several steps. The latter is described in the following.

In order to identify the cell lines which show the most promise of providing a catalytic antibody, it is useful to screen the population for antibodies which bind the transition state analog. This can be accomplished using a standard immunoassay by coupling the transition state analog to a labeled protein which is different both from the carrier molecule used in the immunization and asparagine. The carrier used for screening should be different from both of these molecules to avoid artifacts arising from recognition by the antibodies of epitopes other than the transition state analog mimicked portion of the asparagine. It should be understood, however, that mere binding of the transition state analog is not a demonstration of catalytic activity in the deamidation reaction. Thus, the population of cell lines which produce antibodies that bind the transition state analog should be screened further to demonstrate catalytic activity.

A positive control is measuring the inhibition by the transition state analog of the deamidation of asparagine by the antibody. In order to conclude that the deamidation is in fact caused by catalytic action of the antibody, kinetic measurements of the deamidation process should show that the transition state analog acts as a competitive inhibitor of asparagine.

Upon identification of cell lines which are catalytic deamidating monoclonal antibodies can be characterized and purified from supernatants of cultures of the cells by any of the well-known techniques for accomplishing the purification. For example, the immunoglobulin class can be determined by testing for reaction with anti-IgG or anti-IgM (or other class and sub-class specific) antibodies. If the monoclonal antibody is found to be of the IgG type, it can be purified from the culture supernatants by affinity purification using immobilized protein G or protein A (for example, Bethesda Research Laboratories catalog number 5921SA).

The instant catalytic antibodies can then be used to catalyze the deamidation reaction of asparagine. Also, the instant monoclonal antibodies can be used to catalyze the deamidation of glutamine to glutamic acid.

The present invention also provides for the use of a catalytic antibody as above to use as a therapeutic reagent, for example, in the treatment of cancers such as hematopoietic cancers. For example, such cancers include acute lymphoblastic leukemia, chronic lymphocytic leukemia, Hodgkin's disease, Non-Hodgkin's disease and multiple myeloma. As used herein, “treating cancer” does not necessarily mean curing a patient of cancer. Treating cancer can also include alleviating the symptoms of cancer, reducing tumor load, reducing pain, improving a patient's quality of life, or extending a patient's life expectancy.

Accordingly, further aspects of the invention provide methods of treatment comprising administration of a specific binding member as provided, pharmaceutical compositions comprising such a specific binding member, and use of such a specific binding member in the manufacture of a medicament for administration, for example, in a method of making a medicament or pharmaceutical composition comprising formulating the specific binding member with a pharmaceutically acceptable excipient.

In accordance with the present invention, compositions provided may be administered to individuals. Administration is preferably in a “therapeutically effective amount,” this being sufficient to show benefit to a patient. Such benefit may be at least amelioration of at least one symptom. The actual amount administered, and rate and time-course of administration, will depend on the nature and severity of what is being treated. Prescription of treatment, e.g., decisions on dosage etc, is within the responsibility of general practitioners and other medical doctors. Appropriate doses of antibody are well known in the art; see Ledermann et al. (1991) Int J Cancer 47:659-664; Bagshawe et al. (1991) Antibody, Immunoconjugates, and Radiopharmaceuticals 4:915-922.

A composition may be administered alone or in combination with other treatments, either simultaneously or sequentially dependent upon the condition to be treated.

Pharmaceutical compositions according to the present invention, and for use in accordance with the present invention, may comprise, in addition to active ingredient, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration, which may be oral, or by injection, e.g., intravenous.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous injection, or injection at the site of affliction, the active ingredient will be in the form of a parenterally acceptable aqueous solution which is pyrogen-free and has suitable pH, isotonicity and stability. Those of relevant skill in the art will be able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

The following Examples are supplied to further illustrate the invention. They are not intended to limit the invention in any way.

### EXAMPLE 1

### Synthesis of 2-(acetylamino)-N-(1,4-dihydroxy-1-oxophospolan-3-yl)acetamide

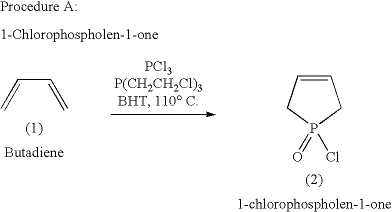

A thick walled glass tube (120 ml capacity) equipped with PCl₃ (17.5 ml, 27.5 g, 0.2 mol), P(OCH₂CH₂Cl)₃ (20.3 ml, 27.0 g, 0.10 mol), 2,6-di-tert-butyl-p-cres (0.22 g, 0.001 mol) and liquid butadiene (condensed from gaseous C₄H₆ at −70° C.; 27 ml, 17 g, 0.32 mol). The glass tube is then sealed with a screw cap and heated to 105° C. using an oil bath. After 19 h, the tube is removed from the oil bath and allowed to cool to room temperature. Filtration of the cloudy yellow solution affords a mixture of 1,2-dichloroethane and the desired 1-chlorophospholen-1-one.

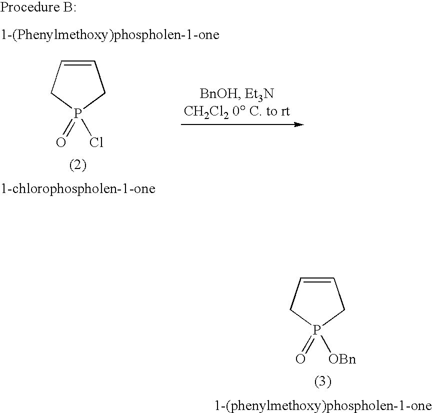

To a rapid stirred solution of PhCH₂OH (4.6 ml, 44.3 mmol) in Et₃N (20 ml) and CH₂Cl₂ (45 ml) at 0° C. is added a portion of the crude 1-chlorophospholen-1-one/1,2-dichloroethane mixture (40 mmol) drop wise slowly via syringe. The resulting suspension is stirred for 12 h at room temperature, concentrated under vacuum, and the resulting white solid is taken up in CH₂Cl₂ (200 ml). The solution is then washed with saturated aqueous NaHCO₃ (3×50 ml), dried (MgSO₄), filtered and concentrated. Purification by flash chromatography 95×15 cm silica gel, 80% EtOH/hexanes) affords 6.8 g (81%) of the desired 1-(phenylmethoxy)phospholen-1-one as a pale yellow oil.

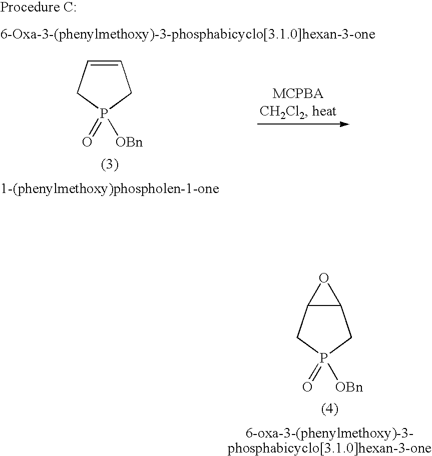

A solution of 1-(phenylmethoxy)phospholen-1-one (227 mg, 0.615 mmol) and m-chlorobenzoic acid (72%, 206 mg, 0.86 mmol) in CH₂Cl₂ (2.5 ml) is refluxed under argon for 24 h, stirred for 12 h at room temperature, and then stirred for 1 h with saturated aqueous NaHCO₃ (˜2 ml). The mixture is then taken up in CH₂Cl₂ (˜100 ml) and washed with saturated NaHCO₃ (3×25 ml) and the combined aqueous layers are then dried (MgSO₄), filtered and concentrated. Purification by flash chromatography (2×15 cm, silica gel, 3% MeOH/CHCl₃) affords 136 mg (57%) of 6-oxa-3-(phenylmethoxy)-3-phosphabicyclo[3.1.0]hexan-3-one.

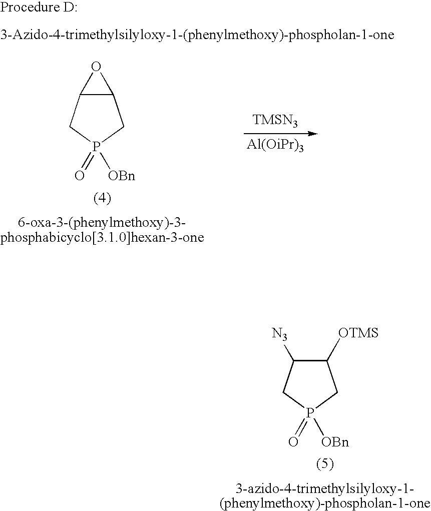

Azidotrimethylsilane (TMSN₃, 0.14 ml, 1.06 mmol), aluminum isopropoxide (Al(OiPr)₃, 22 mg, 0.11 mmol) and CH₂Cl₂ (2.5 ml) are stirred together under Ar for 2 h at room temperature. 6-oxa-3-(phenylmethoxy)-3-phosphabicyclo[3.1.0]hexan-3-one (136 mg 0.353 mmol) is dissolved in CH₂Cl₂ (1 ml+1 ml washing) and added via cannula. After 3 days, the mixture is taken up in CH₂Cl₂ (˜50 ml) and filtered through celite. The solvent is then removed and the crude oil purified by flash chromatography (2×15 cm, silica gel, EtOAc). to afford 70 mg (40%) of 3-azido-4-trimethylsilyloxy-1-(phenylmethoxy)-phospholan-1-one.

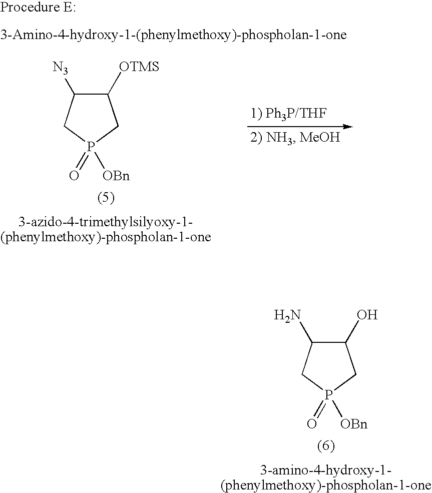

3Azido-4-trimethysilyloxy-1(phenylmethoxy)-phospholan-1one (70 mg, 0.14 mmol) and water (7.6 μL; 0.42 mmol) are dissolved in tetrahydrofuran (1.2 ml). Triphenylphosphine (73 mg, 0.28 mmol) is added and the reaction is stirred ˜14 h at room temperature. The mixture is then concentrated and purified by flash chromatography (2×15 cm silica gel, 20% MeOH/CHCl₃) to afford ˜100 mg (˜100% ) of 3-amino-4-hydroxy-1-(phenylmethoxy)-phospholan-1-one.

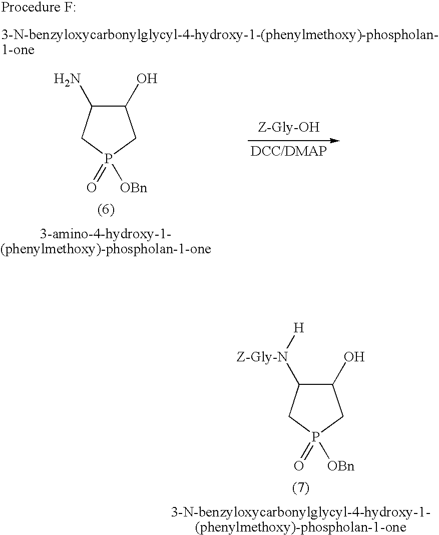

To the solution of 3-amino-4-hydroxy-1-(phenylmethoxy)-phospholan-1-one (100 mg, 0.14 mmol) in CH₂CL₂ (10 ml) are added Z-Gly-OH (29.1 mg, 0.14 mmol), N,N′-Dicyclohexylcarbidiimide (30.3 mg, 14.7 mmol) and 4-dimethylaminopyridine (3.4 mg, 0.028 mmol). The solution is stirred for 12 h. After the precipitate is filtered out, the solution is concentrated under vacuum. Residue is dissolved in EtOAc (30 ml), extracted with saturated NaHCO₃ and citric acid (0.2 M) and saline; dried (MgSO₄). The solvent is then removed to afford the 3-N-benzyloxycarbonylglycyl-4-hydroxy-1-(phenylmethoxy)-phospholan-1-one.

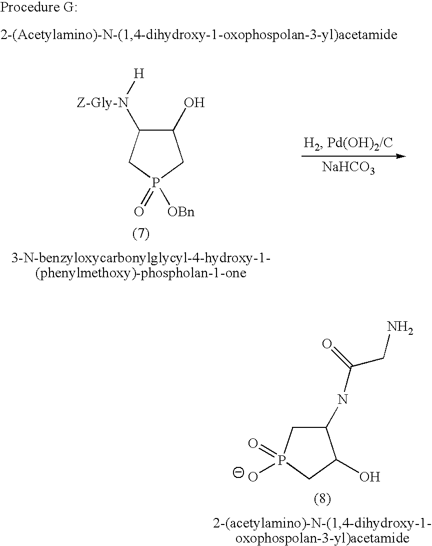

To the solution 3-N-benzyloxycarbonylglycyl-4-hydroxy- 1-(phenylmethoxy)-phospholan-1-one and 10% palladium on activated carbon (20 mg) in THF (20 ml) is added hydrogen continuously. The completion of the reaction is monitored by TLC. After any residue hydrogen is removed by vacuum, the solid is filtered out over the celite. The solvent is then removed to afford the final product, 2-(Acetylamino)-N-(1,4-dihydroxy-1-oxophospolan-3-yl)acetamide.

### EXAMPLE 2

### Synthesis of N-glycinyl-L-phosphonamidylalanine

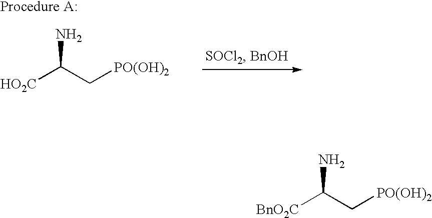

Thionyl chloride (15 mL) was added to the suspension of the D,L-(2-amino-3 phosphono)propionic aid (5 g, 2.96 mmol) in benzyl alcohol (190 mL). The rate of the addition was such to maintain the internal reaction temperature at or below 12° C. (ice/water bath). After the addition was completed, the mixture was allowed to warm-up to room temperature and stirred overnight. The mixture was then concentrated in vacuo (rotoevaporator, bath temp. 90° C.). The thick, white syrup was dispersed in diethyl ether (100 mL). The resulting white solid was collected on a filter, washed with minimal amount of water (10-20 mL) and ether (2×20 mL). The white, soft solid was dried under vacuum in a desiccator for 16 hrs. The yield was 7.1 g (92%).

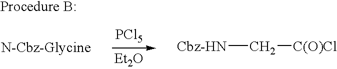

Pure Cbz-Gly-Cl was synthesized using a modified version of literature procedures. Grum-Grzimailo, M. A.; Volkova, L. V.; Serebrennikova, G. A.; Prebrazhenskii, N. A. Zh. Org. Khim. 1967, 3(4), 650-653. Phosphorous pentachloride was added to a suspension of N-Cbz-glycine in anhydrous ether. The mixture was stirred at 0° C. for 50 min. Reaction mixture was then diluted with n-heptane and chilled to −70° C. White solid was collected by filtration, washed with heptane and dried to constant weight under vacuum in a desiccator.

Mp.: 42-43° C. (lit. 40-41° C.).

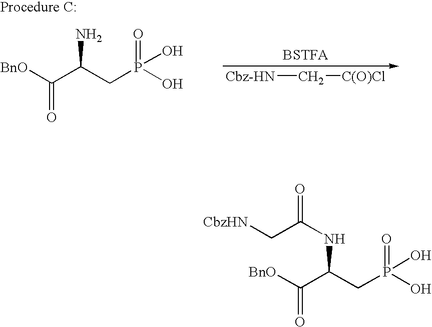

Bis-trimethylsilyltrifluoroacetamide (BSTFA) (5 eq.) is added to the stirred suspension of benzyl 3-phosphonoalanine (0.5 g, 1.93 mmol). The mixture is stirred under nitrogen until clear. N-Cbz-glycine chloride (0.45 g, 1.98 mmol) is then added at 0° C. and the reaction is continued at room temperature for 16-24 hrs. Following concentration under vacuum (rotoevaporator), a light yellow, partially solidified oil (1.2 g, 138%) was obtained. The oil was redissolved in ether (2 mL) and diluted with heptane until no more emulsion was formed. The solvent was collected by decantation and set aside. The remaining oil was concentrated to give 715 mg (82%) of an oil. The heptane extract was concentrated in vacuo to give 460 mg of a pale yellow oil.

The work-up of both fractions was identical and was as follows:

The oil was dissolved in 1 N NaOH (10 mL) and extracted with dichloromethane (3×10 mL). The organic extracts were discarded. The aqueous phase was acidified with 0.5 N HCl to pH between 2 and 3. The oil was extracted with CH₂Cl₂ (3×20 mL). The combined organic extracts were washed with brine (20 mL), dried over MgSO₄, filtered and concentrated.

The oil obtained in each case exhibited identical TLC profile. Overall yield was 592 mg (68%).

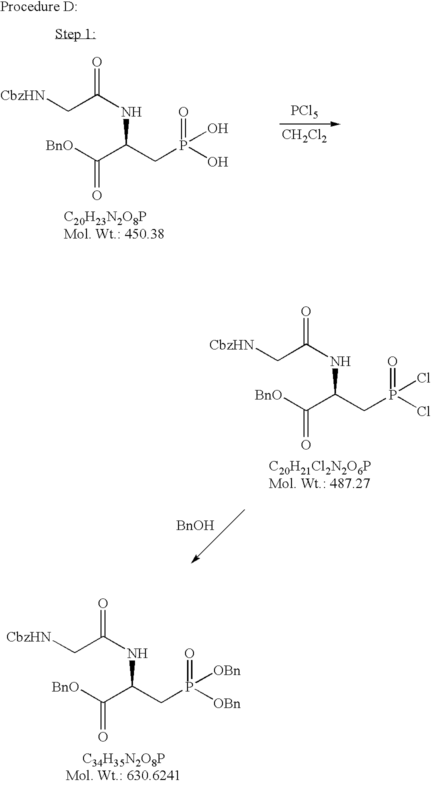

Solid PCl₅ (210 mg, 1 mmol) was added to a solution of the phosphonic acid (450 mg, 1.0 mmol) in dichloromethane (3 mL). An exothern caused solution to boil for a brief moment. The resulting mixture was stirred at RT for 2 hours. The mixture was then concentrated on a rotoevaporator and further dried under high vacuum for 1 hour. The resulting light yellow foam was dissolved in CH₂Cl₂ (3 mL) and was added to a cold solution (0° C.) of benzyl alcohol (6 mmol) and triethylamine (222 mg, 2.2 mmol) in CH₂Cl₂ (6 mL). The mixture was allowed to warm-up slowly to room temperature and stir overnight. The pale yellow solution was concentrated and purified via column chromatography over silica-gel (CH₂Cl₂/MeOH, 98:2) to afford pure bis-benzyl phosphonate as a colorless oil (235 mg, 37%).

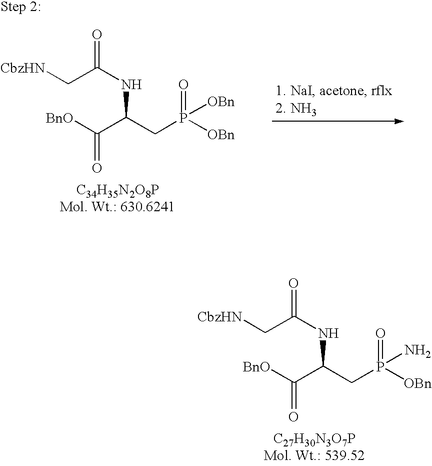

234 mg (0.37 mmol) of di-benzyl phosphonate (mp 85-87° C.) was heated to reflux in acetone (3 mL) with sodium iodide (56 mg, 0.37 mmol), for 4 hours. A white solid precipitated during that time which was collected. The filtrate was left at room temperature overnight. A TLC of the solution revealed presence of the starting material. An additional portion of NaI was added to the solution (40 mg, 0.27 mmol) and the reaction heated to reflux for 4 hours, until complete. Solids that precipitated were collected, washed with acetone, and air dried overnight to afford 155 mg (77%) of the benzyloxyphosphate ester product.

The benzyloxy phosphate ester product was then reacted with ammonia in an inert solvent using standard conditions to afford the benzyloxyphosphonamide product.

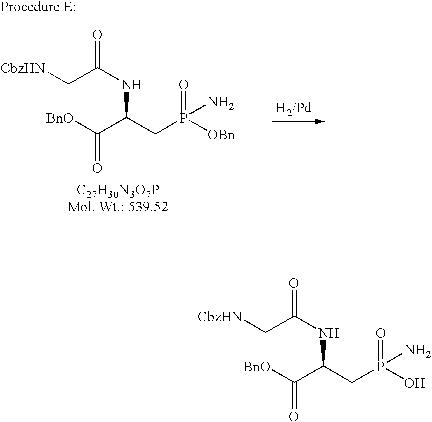

The benzyloxyphosphonamide product with reduced with hydrogen over palladium using standard conditions to afford the title compound, N-glycinyl-L-phosphonamidylalanine.

The foregoing examples are illustrative of certain embodiments of the not intended to be limiting. The scope of the claimed subject he following claims.

1. A compound having the formula: 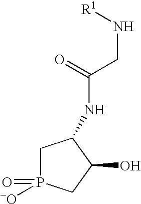
wherein R₁ is a hydrogen atom, an amino protecting group, or a carrier molecule;
and the salts and esters thereof.

2. The compound of claim 1 wherein R₁ is a hydrogen atom.

3. The compound of claim 1 wherein R₁ is an amino protecting group.

4. The compound of claim 1 wherein R₁ is a carrier molecule.

5. A monoclonal catalytic antibody specific for a transition state analog of claim 1 that catalyzes the deamidation of asparagine to aspartic acid.

6. The monoclonal antibody of claim 5 that was raised against the transition state analog of claim 1 wherein R₁ is an immunoconjugate carrier molecule.

7. The monoclonal antibody of claim 5 wherein the antibody is murine.

8. The monoclonal antibody of claim 5 wherein the antibody is human.

9. The monoclonal antibody of claim 5, wherein the antibody is a human one of the IgG class.

10. The monoclonal antibody of claim 5, wherein the antibody is a human one of the IgG₂ class.

11. A pharmaceutical composition comprising a pharmaceutically acceptable amount of the monoclonal antibody of claim 5 and one or more pharmaceutically acceptable carriers therefor.

12. The pharmaceutical composition of claim 11, wherein the monoclonal antibody is murine.

13. The pharmaceutical composition of claim 11, wherein the monoclonal antibody is human.

14. The pharmaceutical composition of claim 11, wherein the monoclonal antibody is a human one of the IgG class.

15. The pharmaceutical composition of claim 11, wherein the monoclonal antibody is a human one of the IgG₂ class.

16. A method of treating cancer which comprises administering to a patient in need of such treatment a pharmaceutically effective amount of the pharmaceutical composition of claim 11.

17. The method of claim 16, where in the monoclonal antibody of the pharmaceutical composition is a murine one.

18. The method of claim 16, where in the monoclonal antibody of the pharmaceutical composition is a human one.

19. The method of claim 16, where in the monoclonal antibody of the pharmaceutical composition is a human one of the IgG class.

20. The method of claim 16, where in the monoclonal antibody of the pharmaceutical composition is a human one of the IgG₂ class.

21. The method of claim 16, wherein the cancer to be treated is a hematopoietic cancer.

22. The method of claim 16, wherein the cancer to be treated is acute lymphoblastic leukemia.

23. The method of claim 16, wherein the cancer to be treated is acute lymphoblastic leukemia.

24. The method of claim 16, wherein the cancer to be treated is chronic lymphocytic leukemia.

25. The method of claim 16, wherein the cancer to be treated is Hodgkin's disease.

26. The method of claim 16, wherein the cancer to be treated is Non-Hodgkin's lymphoma.

27. The method of claim 16, wherein the cancer to be treated is multiple myeloma.

28. A method of catalyzing the deamidation of asparagine to aspartic acid comprising contacting asparagine-containing material with an antibody of claim 5.

29. A method of expressing an antibody of claim 5 that comprises immunizing a mouse with a compound of claim 1, forming a hybridoma from the spleen of the mouse so immunized, and isolating the antibody expressed from the hybridoma.

30. A compound having the formula: 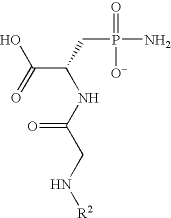
wherein R₂ is a hydrogen atom, an amino protecting group, or a carrier molecule;
and the salts and esters thereof.

31. The compound of claim 30 wherein R₂ is a hydrogen atom.

32. The compound of claim 30 wherein R₂ is an amino protecting group.

33. The compound of claim 30 wherein R₂ is a carrier molecule.

34. A monoclonal catalytic antibody specific for a transition state analog of claim 30 that catalyzes the deamidation of asparagine to aspartic acid.

35. The monoclonal antibody of claim 34 that was raised against the transition state analog of claim 30 wherein R₂ is an immunoconjugate carrier molecule.

36. The monoclonal antibody of claim 34 wherein the antibody is murine.

37. The monoclonal antibody of claim 34 wherein the antibody is human.

38. The monoclonal antibody of claim 34, wherein the antibody is a human one of the IgG class.

39. The monoclonal antibody of claim 34, wherein the antibody is a human one of the IgG₂ class.

40. A pharmaceutical composition comprising a pharmaceutically acceptable amount of the monoclonal antibody of claim 34 and one or more pharmaceutically acceptable carriers therefor.

41. The pharmaceutical composition of claim 40, wherein the monoclonal antibody is murine.

42. The pharmaceutical composition of claim 40, wherein the monoclonal antibody is human.

43. The pharmaceutical composition of claim 40, wherein the monoclonal antibody is a human one of the IgG class.

44. The pharmaceutical composition of claim 40, wherein the monoclonal antibody is a human one of the IgG₂ class.

45. A method of treating cancer which comprises administering to a patient in need of such treatment a pharmaceutically effective amount of the pharmaceutical composition of claim 40.

46. The method of claim 45, where in the monoclonal antibody of the pharmaceutical composition is a murine one.

47. The method of claim 45, where in the monoclonal antibody of the pharmaceutical composition is a human one.

48. The method of claim 45, where in the monoclonal antibody of the pharmaceutical composition is a human one of the IgG class.

49. The method of claim 45, where in the monoclonal antibody of the pharmaceutical composition is a human one of the IgG₂ class.

50. The method of claim 45, wherein the cancer to be treated is a hematopoietic cancer.

51. The method of claim 45, wherein the cancer to be treated is acute lymphoblastic leukemia.

52. The method of claim 45, wherein the cancer to be treated is acute lymphoblastic leukemia.

53. The method of claim 45, wherein the cancer to be treated is chronic lymphocytic leukemia.

54. The method of claim 45, wherein the cancer to be treated is Hodgkin's disease.

55. The method of claim 45, wherein the cancer to be treated is Non-Hodgkin's lymphoma.

56. The method of claim 45, wherein the cancer to be treated is multiple myeloma.

57. A method of catalyzing the deamidation of asparagine to aspartic acid comprising contacting asparagine-containing material with an antibody of claim 34.

58. A method of expressing an antibody of claim 34 that comprises immunizing a mouse with a compound of claim 30, forming a hybridoma from the spleen of the mouse so immunized, and isolating the antibody expressed from the hybridoma.
